(19) United States
(12) Patent Application Publication
(10) Pub. No.: US 2023/0003367 A1
(43) Pub. Date: Jan. 5, 2023

(54) UV CEILING LIGHT

(71) Applicant: SHENZHEN GUANKE TECHNOLOGIES CO., LTD, Shenzhen (CN)

(72) Inventors: Qing Lan, Shenzhen (CN); Shoubao Chen, Shenzhen (CN); Ligen Liu, Shenzhen (CN); Nengsheng Chen, Shenzhen (CN)

(73) Assignee: SHENZHEN GUANKE TECHNOLOGIES CO., LTD, Shenzhen (CN)

(21) Appl. No.: 17/412,636

(22) Filed: Aug. 26, 2021

(30) Foreign Application Priority Data
Jun. 30, 2021 (CN) 2021214936667

(51) Int. Cl. F21V 21/04; F21V 29/76; F21V 29/67; F21S 8/02

(52) U.S. Cl. CPC F21V 21/047; F21V 29/763; F21V 29/677; F21S 8/026; A61L 9/20

(57) ABSTRACT

A UV ceiling light including a mounting box, a mounting board, driving power supply, a UV module, a grating component and a cover plate. There is a mounting space inside the mounting box, the bottom of the mounting box is provided with avoiding holes connecting to the mounting space, the mounting board is provided on the upper end of the mounting box in a dismountable way, and the driving power supply is provided inside the mounting space; the upper end of the UV module passes through the avoiding hole and extends into the mounting space, and the UV module electrically connects to the driving power supply; the grating component is provided on the lower surface of the mounting box, the grating component includes several separating plates that are horizontally circular and provided around the UV module, and each two adjacent separating plates form a light outlet in an enclosing way.

### BACKGROUND

### Technical Field

The present invention relates to the technical field of lights, particularly to a UV ceiling light.

### Background

The power of strong-UV lights is usually greater than 20 W. The strong UV light emitted by such lights can effectively kill bacteria and virus and shorten the sterilization time, so such UV lights achieve a good sterilization and disinfection effect. However, to ensure the safety performance, strong-UV lights in relevant technologies are usually designed into large-size cabinets, so such strong-UV lights occupy a large space and are relatively heavy, which brings installation difficulties and higher costs.

The foregoing content is only used for assisting in understanding the technical scheme of this invention, but does not mean the acknowledgement of that the above content is the current technology.

### SUMMARY

The main objective of the present invention is to provide a UV ceiling light to improve the installation convenience and use safety of UV lights and reduce the manufacture cost of UV lights.

To realize the above objective, the UV ceiling light in the present invention comprises:

a mounting box and a mounting board, defining that the UV ceiling light is of a top-bottom structure, wherein there is a mounting space inside the mounting box, the bottom of the mounting box is also provided with avoiding holes connecting to the mounting space, the mounting board is provided on the upper end of the mounting box in a dismountable way, the mounting board is used for external installation of the UV ceiling light;

driving power supply, provided in the mounting space;

UV module electrically connecting to the driving power supply, with its upper end passes through the avoiding holes and extends into the mounting space;

a grating component provided on the lower surface of the mounting box, including several separating plates provided at intervals from the top to the bottom, wherein several the separating plates are circular and are provided around the UV module horizontally, each two adjacent the separating plates form a light outlet in an enclosing way, and UV light emitted by the UV module is emitted horizontally via the light outlets; and

a cover plate below the grating component.

Other characteristics and corresponding beneficial effects of the present invention are elaborated in the latter part of the description.

The solving ideas of technical problems of the present invention and relevant product design solutions are as shown below: The strong-UV light is designed into a ceiling light structure. By making advantage of the installation characteristics of the ceiling light, the UV ceiling light in the present invention can be installed in the upper space more than 2.1 m above the ground for use. Thus, the UV ceiling light comprises a mounting board and a mounting box, wherein the mounting board mounting board is used for external installation of the ceiling light; the mounting box, as the main body of the light, connects to the mounting board in a dismountable way.

Different from ordinary lighting ceiling lights, the light source module of this UV ceiling light is not provided inside the mounting box, but the illuminating part of the UV module is provided at the bottom of the mounting box, so that the illuminating part can be exposed for sterilization, and the light cap (on the upper end of the UV module) and the driving power supply of the UV module are provided inside the mounting box.

In addition, the periphery of the exposed illuminating part of the UV module is provided with a grating component, and the horizontal baffles of the grating component limit that the UV light emitted by the UV module only goes out horizontally. In such a case, horizontally emitted UV light can directly contact air outside the light to carry out sterilization and improve the sterilization efficiency; and, the UV light can be limited in the upper space, preventing damage to human bodies.

Furthermore, the grating component can adopt the sheet stamping process to significantly reduce the weight of the grating component. In the meanwhile, baffles of the grating component are designed into circles to form a central hole where the UV module is located, which significantly decrease the size and weight of the light. Additionally, under the premise of guaranteeing the safety, the cost of the strong-UV light is reduced too. Taking 30 W UV downlight as an example, the size of the present invention can be decreased to φ170×1250 MM, enabling a compact structure, and its weight can be decreased to less than 1.7 Kg; however, the size of cabinet strong-UV lights in relevant technologies is more than 0.6 m or 1 m, so they are heavy and it is hard that they are installed by an individual person. Thus, the UV ceiling light in the present invention is characterized by a small size, light weight, low manufacture cost, convenient installation and safe use.

### BRIEF DESCRIPTION OF THE DRAWINGS

FIG. 1 is a schematic diagram showing the structure of an example of the UV light of the present invention;

FIG. 2 is a schematic diagram showing the structure of another perspective of the UV ceiling light in FIG. 1;

FIG. 3 is a schematic diagram showing the structure of the UV ceiling light in FIG. 1 after removing the mounting board and the protective cover;

FIG. 4 is a schematic diagram showing the explosive structure of the UV ceiling light in FIG. 1 from one perspective;

FIG. 5 is a schematic diagram showing the explosive structure of the UV ceiling light in FIG. 1 from another perspective;

FIG. 6 is a schematic diagram showing the cross section of the UV ceiling light in FIG. 1;

FIG. 7 is a schematic diagram showing the explosive structure of the mounting box and the mounting board of the UV ceiling light in FIG. 1;

FIG. 8 is a schematic diagram showing the structure of the grating component of the UV ceiling light in FIG. 1.

The shape, dimension, proportion or position relationship of parts of the product in drawings may be real data of embodiments and they are under protection of this invention.

### DETAILED DESCRIPTION OF THE EMBODIMENTS

To make the objective, technical solutions and advantages of the present invention clearer and be understood better, further detailed descriptions of examples of the present invention are made in combination with drawings. Understandably, the specific examples described are just used to explain but not limit the present invention.

The present invention provides a UV ceiling light.

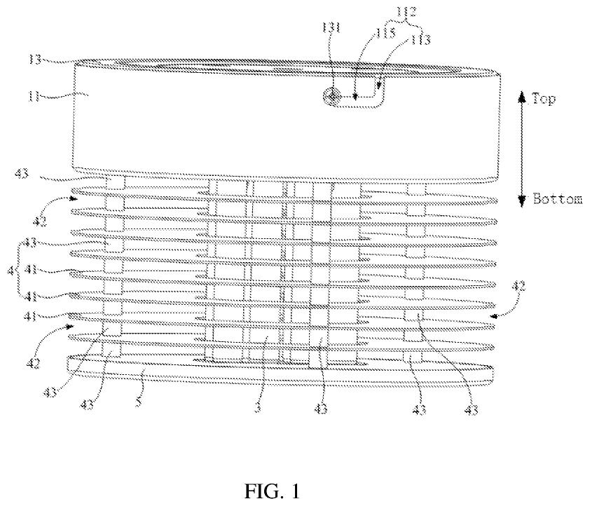

In one example of the present invention, as shown in FIGS. 1-6, the UV ceiling light comprises a mounting box 11, a mounting board 13, driving power supply 2, a UV module 3, a grating component 4 and a cover plate 5. Wherein, defining that the UV ceiling light is of a top-bottom structure, there is a mounting space 110 inside the mounting box 11, the bottom of the mounting box 11 is provided with avoiding holes 111 connecting to the mounting space 110, the mounting board 13 is provided on the upper end of the mounting box 11 in a dismountable way, the mounting board 13 is used for external installation of the UV ceiling light; the driving power supply 2 is provided inside the mounting space 110; the upper end of the UV module 3 passes through the avoiding holes 111 and extends into the mounting space 110, and the UV module 3 electrically connects to the driving power supply 2; the grating component 4 is provided on the lower surface of the mounting box 11, comprises several separating plates 41 provided at intervals from the top to the bottom, several separating plates 41 are circular (e.g. circular shape, square shape or other enclosing-type geometric shapes containing a central hole) and are provided around the UV module 3 horizontally, each two adjacent separating plates 41 from a light outlet 42 in an enclosing way, UV light emitted by the UV module 3 is emitted horizontally via the light outlets; the cover plate 5 is below the grating component 4.

In one example of the present invention, the UV ceiling light can connect other devices via the mounting board 13. For example, the mounting board 13 can connect to a roof, wall or other supporting surface directly or via a suspending pole, so that the UV ceiling light can be installed in the upper space.

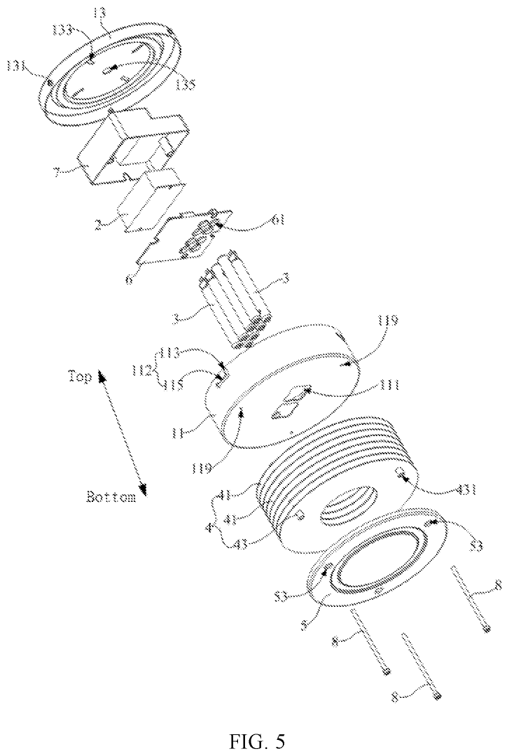

The UV module 3 can be a gas discharge lamp tube composed of several hollow tubes connected in sequence, and the length of the hollow tubes is vertical to the separating plates 41. Since UV lights with gas discharge UV lamp tubes, such as low-pressure mercury lamp, high and medium-pressure mercury lamp and xenon lamp, have high UV conversion efficiency, the sterilization effect of the UV ceiling light can be improved. The lamp tube is composed of several hollow tubes connected in sequence, which can increase the effective length of the lamp tube and then increase the power of the lamp tube; and shorten the length of the tube. In this example, as shown in FIG. 5, the light adopts two 15 W lamp tubes, each lamp tube composes four parallel straight tubes connected in sequence, enabling the tube length to decrease by about 4 times; the tube can also be composed of two U-shaped tubes. After the tube length is shortened, the quantity of separating plates 41 used can be reduced, the ceiling light is lighter and has a more compact structure. Furthermore, the length of the hollow tubes is vertical to the separating plates 41, enabling UV light to be emitted via the light outlets more easily and reducing energy loss. Additionally, compared with lights which adopt one hollow tube, the light which adopts several hollow tubes enables the intensity of UV light emitted via the same light outlet 41 to increase by times, so that the emission distance of UV light via the light outlets will be further and the sterilization scope of the light will be improved.

The UV module 3 can also adopt the UV LED which comprises UV LED lamp beads and a strip-shaped heat radiator. In such a case, the UV LED lamp beads are provided on the periphery of the heat radiator corresponding to light outlets 42 (that is, LED lamp beads are provided between two adjacent separating plates 41 to make UV light emitted by LED be directly emitted via light outlets and reduce loss of UV light caused by separating plates), and the length of the heat radiator is vertical to the separating plates.

The cover plate 5 can be used for fixing grating component 5, mounting box 11 and UV module. To improve the light shielding effect and the locating effect of the cover plate 5 for the UV module 3, the position on the cover plate 5 corresponding to the lower end of the UV module 3 can be provided with a groove 51, and the lower part of the UV module 3 is against the inner side of the groove 51. The groove 51 can adopt the flexible plastic, or can be coated with flexible materials, or can be composed with flexible parts. For example, an independent flexible plastic part or elastic part can be inserted in the groove. When the UV module 3 is a lamp tube, since most tubes are fragile (e.g. quartz), lamp tubes usually need to be fixed with glue, and the glue curing process is involved during production and assembly. Since flexible grooves 51 are preset, the lamp tube can be fixed directly with glue when assembling the ceiling light, without the need of waiting for curing of glue before assembly of the whole ceiling light. So, a lot of assembly time is saved and the productivity of lights is improved.

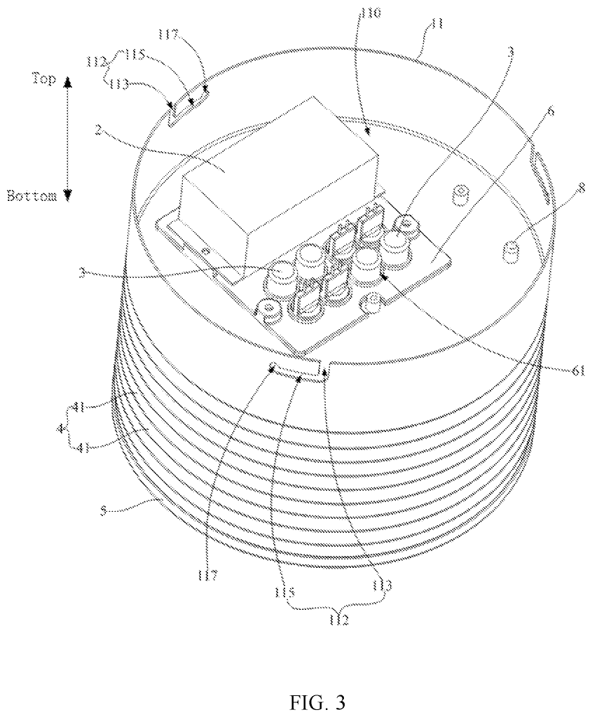

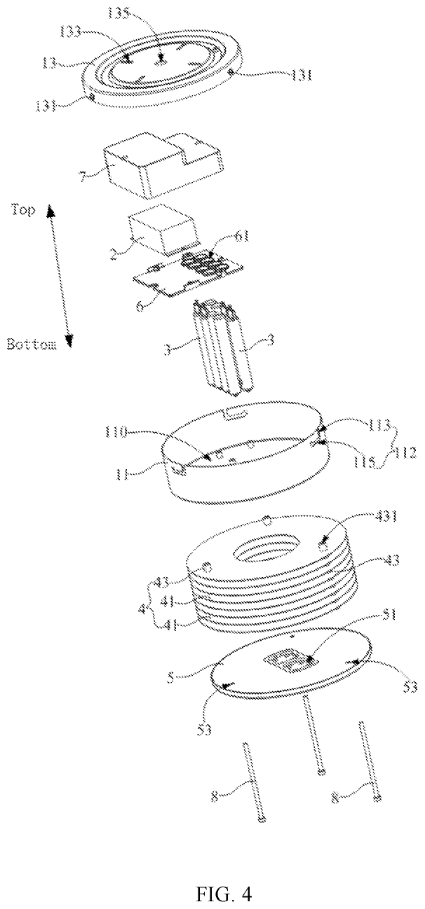

In one example of the present invention, as shown in FIG. 3 and FIG. 4, the UV ceiling light also comprises a connecting fitting 6 provided inside the mounting space 110 and connecting to the mounting box 11 in a dismountable way; at least one installation position 61 is formed at the position on the connecting fitting 6 corresponding to the avoiding holes 111, there should be at least one UV module 3 fixed onto the installation position 61. As shown in FIGS. 1-5, the UV ceiling light is provided with two 15 W UV modules 3.

Understandably, the UV module 3 is not directly fixed onto the mounting box 11, but adopts a connecting fitting 6. The connecting fitting 6 is provided with installation positions 61 to facilitate this UV module 3 to be installed onto the connecting fitting 6, and the connecting fitting 6 connects to the mounting box 11 in a dismountable way. As a result, when the UV module 3 breaks down, the connecting fitting 6 can be removed from the mounting box 11 to repair and replace the UV module 3. And, via this connecting fitting 6, one or several installation positions 61 can be set according to the demand for the power of the ceiling light, which facilitates extension of several UV modules 3, derivation of different power types of ceiling lights and realization of series of products. Wherein, the connecting fitting 6 can be roughly a plate-shaped structure and can be provided with a through-hole to form an installation position 61. Of course, the connecting fitting 6 can also be of a frame structure containing two clamping arms, and the space between two clamping arms forms an installation position 61. In such a case, the upper end of the UV module 3 is clamped by two clamping arms. The connecting fitting 6 and the mounting box 11 can be connected via screws, snap joints or magnet.

In one example of the present invention, as shown in FIG. 3 and FIG. 4, the connecting fitting 6 is made of insulation materials, and the driving power supply 2 is provided on the surface of the connecting fitting 6 departing from the mounting space 110.

Understandably, the driving power supply 2 is also provided on the connecting fitting 6, so that it can be close to the UV module 3, and the driving power supply 2 and the UV module 3 can be electrically connected. In the meanwhile, such design enables them to be more compact inside the mounting space 110 and then decreases the overall size of the mounting box 11. In this example, the connecting fitting 6 is of a square plate structure provided at the bottom of the mounting box 11.

In one example of the present invention, as shown in FIG. 4 and FIG. 5, the UV ceiling light also comprises a protective cover 7 provided inside the mounting space 110 and connecting to the connecting fitting 6 or the mounting box 11 in a dismountable way, and the protective cover 7 also covers the connecting fitting 6, the UV module 3 and the driving power supply 2.

Understandably, by covering the connecting fitting 6, the UV module 3 and the driving power supply 2 with the protective cover 7, electric shock and exposure of UV light can be prevented, so as to improve the use safety of the light. Wherein, the protective cover 7 and the connecting fitting 6 or the mounting box 11 can be connected via screws, snap joints of magnet.

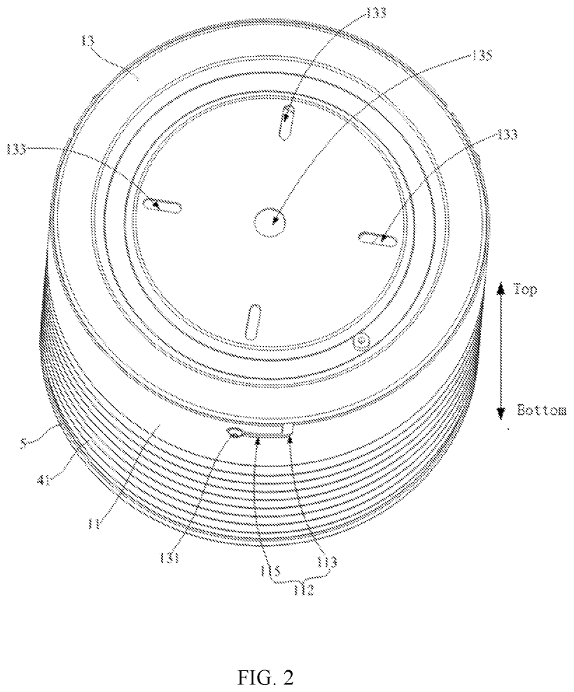

In one example of the present invention, as shown in FIG. 2 and FIG. 4, on the projection plane vertical to the top-bottom direction, the projection of the mounting box 11, the mounting board 13, the grating component 4 and the cover plate 5 is circular.

In such a case, the UV light emission distance can be shortened, and the sterilization efficiency can be improved. In the meanwhile, the periphery of the light is of consistency, preventing limitation of the installation direction of the light; reducing the volume of the light and enabling a compact structure. Of course, the present invention is not limited to the above solutions. On the projection plane vertical to the top-bottom direction, the mounting box 11, the mounting board 13, grating component 4 and cover plate 5 can be square or other shapes.

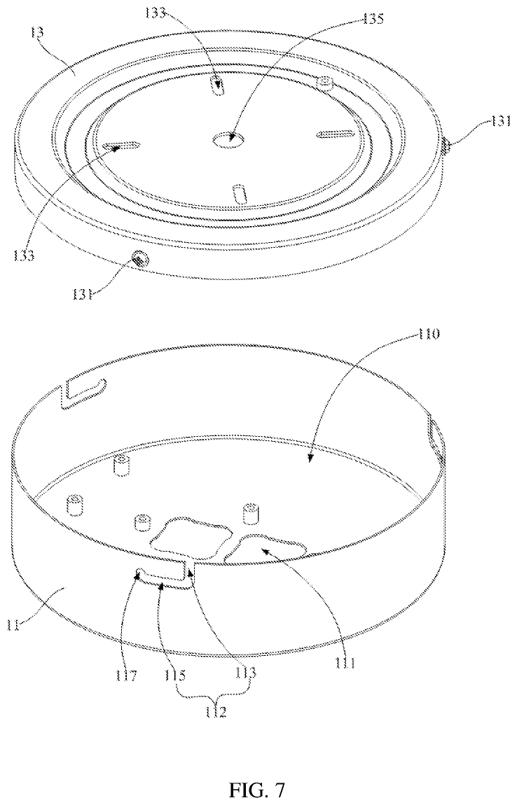

In one example of the present invention, as shown in FIG. 1, FIG. 2 and FIG. 7, one part of mounting board 13 extends into the mounting space 110, the mounting board 13 which extends into the mounting space 110 is provided with protruding clamping columns 131, the position on the lateral periphery of mounting box 11 corresponding to the clamping columns 131 is provided with grooves 112, the grooves 112 comprise the first groove section 113 and the second grove section 115, the first groove section 113 extends along the top-bottom direction and passes through the upper surface of the mounting box 11, the second grove section 115 extends along the horizontal direction vertical to the up direction and connects to the lower end of the first groove section 113, the second grove section 115 forms a locating groove 117 corresponding to the upper one of two groove side walls, the clamping columns 131 are embedded into the locating grooves 117 to make the mounting board 13 connect to the mounting box 11 in a dismountable way. The upper surface of the mounting board 13 is also provided with several strip-shaped mounting holes 133 connecting to the mounting space 110 and one cord hole 135, several mounting holes 133 are distributed at intervals around the center of the mounting board 13, and the cord hole 135 is in the center of the mounting board 13.

Understandably, by making the clamping columns 131 pass through the first groove section 113 and the second grove section 115 in sequence and then making it be clamped in the locating grooves 117, the mounting board 13 and the mounting box 11 can be connected to improve the dismounting convenience of the mounting board 13 and the mounting box 11. And, locating grooves 117 can limit the location of the clamping columns 131 in the top-bottom direction and the second grove section 115 in the extension direction, reducing the possibility of looseness of the clamping columns 131. With the mounting holes 133, the mounting board can be installed by using a suspending pole to improve the convenience of external installation. Mounting holes 133 are in the strip shape, which enables the installation position to be adjusted to some extent. The cord hole 135 facilitates that the cord can be laid orderly to connect to the driving power supply 2. Additionally, it needs to be explained that in other examples of the present invention, the mounting board 13 can directly connect to the mounting box 11 via screws.

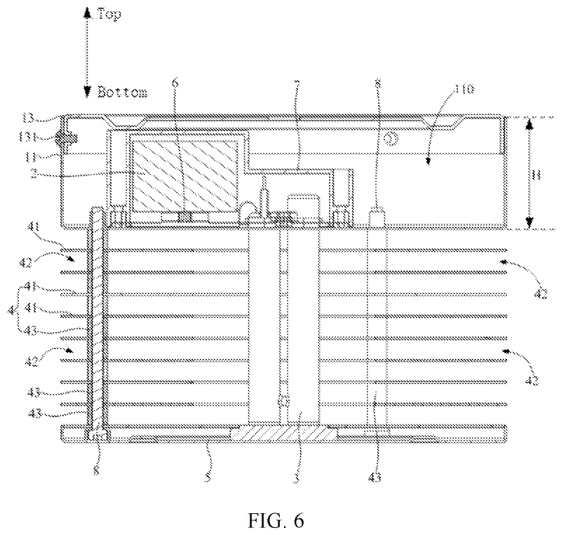

In one example of the present invention, as shown in FIG. 6, the height of the mounting box 11 is H (2 mm≤H≤20 mm). In this case, there is a certain distance between the mounting box 11 and the surface used for installing this UV ceiling light to reduce the possibility that UV light is emitted onto the surface used for installing the UV ceiling light, preventing damage to the surface used for installing the UV ceiling light caused by illumination of the UV light. Furthermore, on the projection plane vertical to the top-bottom direction, the projection area of the mounting box 11 can be greater or equal to the projection area of several separating plates 41. In such a case, the mounting box 11 can better shield UV light emitted by the light to reduce the possibility that UV light is emitted onto the surface for installing the UV ceiling light.

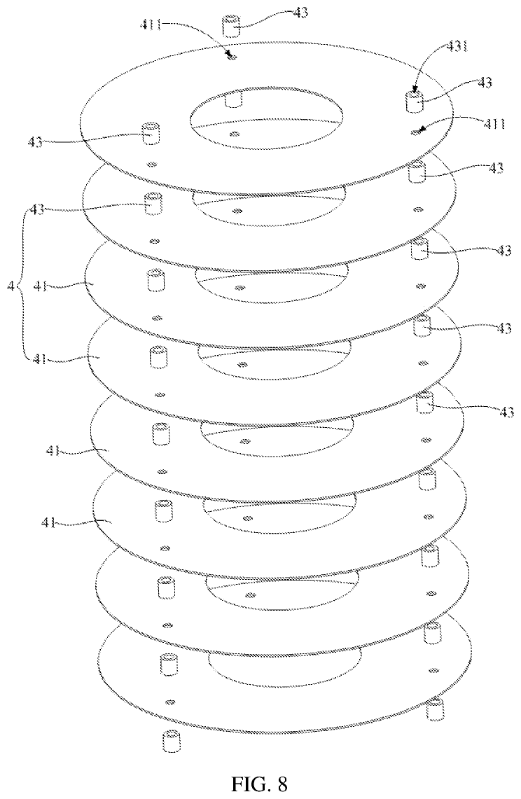

In one example of the present invention, as shown in FIG. 1, FIG. 4, FIG. 5, FIG. 6 and FIG. 8, the UV ceiling light also comprises fasteners 8, and the grating component 4 also comprises several connecting poles 43. The space between the lower surface of the mounting box 11 and the separating plates 41, the space between adjacent separating plates 41 and the space between the separating plates 41 and the upper surface of the cover plate 5 are corresponding to several layers respectively, the connecting poles 43 are provided among layers in a clamping way to limit the height of each layer and form several light outlets 42. Each layer can be provided with several connecting poles 43 which are provided at intervals around the periphery of separating plates 41. The shaft center of the connecting poles 43 is provided with the first through-hole 431, the separating plates 41 are provided with the third through-hole 411 corresponding to the first through-hole 431, the lower surface of the mounting box 11 is provided with fixing holes 119 corresponding to the first through-hole 431, and the position on the cover plate 5 corresponding to the first through-hole 431 is provided with the second through-hole 53. Several adjacent connecting poles 43 compose a group of connecting poles, that is, the grating component 4 can comprise several groups of connecting poles provided at intervals around the periphery of the separating plates 41. For the same group of connecting poles, the fixing holes 119, the first through-hole 431, the third through-hole 411 and the second through-hole 53 are connected mutually, which enables fasteners 8 to be inserted into the fixing holes 119, the first through-hole 431, the third through-hole 411 and the second through-hole 53 to realize assembly and dismountable connection of the cover plate 5, grating component 4 and mounting box 11.

Since the UV ceiling light is small, and the structural strength of the ceiling light is improved by using the connecting fitting 6, the separating plates 41, cover plate 5 and mounting box 11 can adopt the sheet stamping process to significantly reduce the weight and cost of the light.

Additionally, cover plate 5 and grating component 4 connect to mounting box 11 in a dismountable way, so that when one of them is damaged, they can be dismounted directly to repair or replace the damaged one. With the fixing holes 119, the first through-hole 431, the third through-hole 411 and the second through-hole 53, the cover plate 5, the grating component 4 and the mounting box 11 can be installed at one time by making the cord pass through the fasteners 8, which significantly improves their installation efficiency. Wherein, the fixing holes 119 can be threaded holes, and the fasteners 8 can be screws. Screw fastening is simple and reliable, so screw fastening can guarantee the connection stability of the cover plate 5, grating component 4 and mounting box 11, simplify their connection structure and then improve their dismounting convenience. Of course, the fixing holes 119 can also be snap joint holes, and, in such a case, the fasteners 8 are clamping columns, enabling the fasteners 8 to be snap-fitted with the fixing holes 119. And, it needs to be explained that any two of the cover plate 5, grating component 4 and mounting box 11 can be connected independently.

In one example of the present invention, the UV ceiling light also comprises a fan provided at the bottom of the cover plate 5. The fan blows air downward vertically. The air inlet of the fan can connect to the mounting space of the UV module 3, can connect to light outlets 42 and can also connect to the peripheral space of the cover plate 5 to make sterilized air in the surrounding area of the grating component 4 be inhaled into the fan sufficiently, and then the air is blown by the fan to the lower space (usually the space below 2.1 m). The fan can exhaust sterilized air in the upper space better to promote ventilation of air in the upper space and the lower space and expand the sterilization scope of the UV lights for the surrounding air. In such a way, the UV light can directly sterilize and disinfect horizontal air corresponding to the grating component 4, and horizontal air and vertical air can be convected well too to realize indirect sterilization and disinfection of the air in areas beyond the horizontal layer corresponding to the grating component 4.

The above description only presents the preferred examples of the invention, and it is not for this reason that the patent scope of the invention is limited. Any equivalent structural transformation made by using the description of the invention and the drawings, or direct/indirect application in other related technical fields under the inventive concept of the invention, is included in the patent protection scope of the invention.

What is claimed is:

1. A UV ceiling light comprising:
a mounting box and a mounting board, defining the UV ceiling light as a top-bottom structure, wherein there is a mounting space inside the mounting box, a bottom of the mounting box is provided with avoiding holes connecting to the mounting space, the mounting board is provided on an upper end of the mounting box in a dismountable way, the mounting board is used for external installation of the UV ceiling light;
a driving power supply, provided in the mounting space;
a UV module electrically connecting to the driving power supply, with an upper end of the UC module passing through the avoiding holes and extending into the mounting space;
a grating component provided on a lower surface of the mounting box, including several separating plates provided at intervals from a top to a bottom, wherein several of the separating plates are circular and are provided around the UV module horizontally, each two adjacent separating plates form a light outlet in an enclosing way, and UV light emitted by the UV module is emitted horizontally via light outlets; and
a cover plate below the grating component.

2. The UV ceiling light as claimed in claim 1, wherein the UV module is a gas discharge light tube comprised of several hollow tubes connected in sequence, a length of the hollow tubes is vertical to the separating plates;
or the UV module comprises a UV LED and a strip-shaped heat radiator, the UV LED is provided on a periphery of the heat radiator corresponding to the light outlets, the length of the heat radiator is vertical to the separating plates.

3. The UV ceiling light as claimed in claim 1, wherein the UV lights further comprise a connecting fitting provided inside the mounting space and connecting to the mounting box in a dismountable way; and
a position on the connecting fitting corresponding to the avoiding holes is provided with at least one installation position, and there is at least one UV module fixed to the installation position.

4. The UV ceiling light as claimed in claim 3, wherein the UV lights also comprise a protective cover provided inside the mounting space and connecting to the connecting fitting or the mounting box in a dismountable way, the protective cover covers the connecting fitting, and the UV module and the driving power supply;
and/or, the connecting fitting is made of insulation materials, the driving power supply is provided on the surface of the connecting fitting departing from the bottom wall of the mounting space.

5. The UV ceiling light as claimed in claim 1, wherein, on the projection plane vertical to the top-bottom direction, the projection of the mounting box, the mounting board, the grating component, and the cover plate is circular.

6. The UV ceiling light as claimed in claim 5, wherein part of the mounting board extends into the mounting space, the mounting board which extends into the mounting space is provided with protruding clamping columns, the lateral peripheral wall of the mounting box is provided with grooves corresponding to the clamping columns, the grooves comprise the first groove section and the second grove section, the first grove section is provided in the top-bottom direction and passes through the upper surface of the mounting box, the second grove section is provided along the horizontal direction vertical to the up direction and connects to the lower end of the first section, the second grove section forms a locating groove corresponding to the upper one of two groove side walls, the clamping columns are embedded into the locating grooves to make the mounting board connect to the mounting box in a dismountable way,
the upper surface of the mounting board is also provided with several strip-shaped mounting holes connecting to the mounting space and one cord hole, several the mounting holes are distributed at intervals around the center of the mounting board, and the cord hole is in the center of the mounting board.

7. The UV ceiling light as claimed in claim 1, wherein, on the projection plane vertical to the top-bottom direction, the projection of the mounting box is greater than or equal to that of several separating plates;
and/or, the height of the mounting box is H, 20 mm≤H≤200 mm.

8. The UV ceiling light as claimed in claim 1, wherein the UV ceiling light further comprises fasteners, and the grating component also comprises several connecting poles;
the space between the lower surface of the mounting box and the separating plates, the space between adjacent the separating plates and the space between the separating plates and the upper surface of the cover plate correspond to several layers respectively, the connecting poles are provided among layers in a clamping way to form several the light outlets;
the axis of the connecting poles is provided with a first through-hole, the separating plates are provided with a third through-hole corresponding to the first through-hole, the lower surface of the mounting box is provided with fixing holes corresponding to the position of the first through-hole, and the cover plate is provided with a second through-hole corresponding to the position of the first through-hole;
the fasteners are inserted into the fixing holes, the first through-hole, the third through-hole and the second through-hole to realize assembly of the cover plate, the grating component and the mounting box.

9. The UV ceiling light as claimed in claim 1, wherein the cover plate is provided with a groove corresponding to the lower end of the UV module, the lower end of the UV module is against the inner side of the groove; and the groove is composed of elastic parts, or the surface of the groove is made of soft materials.

10. The UV ceiling light as claimed in claim 1, wherein the UV ceiling light further comprises a fan provided at the bottom of the cover plate, and the fan blows air downward vertically.
